(19) United States
(12) Patent Application Publication
(10) Pub. No.: US 2008/0188720 A1
(43) Pub. Date: Aug. 7, 2008

(54) System and method for predicting osteoarthritis pain based on change in barometric pressure, and ambient temperature

(75) Inventors: Timothy E. McAlindon, Belmont, MA (US); Christopher H. Schmid, Natick, MA (US)

Correspondence Address: NIXON PEABODY, LLP, 401 9TH STREET, NW, SUITE 900, WASHINGTON, DC, 20004-2128, US

(73) Assignee: New England Medical Center, Boston, MA (US)

(21) Appl. No.: 11/878,635

(22) Filed: Jul. 25, 2007

Related U.S. Application Data
(60) Provisional application No. 60/820,358, filed on Jul. 26, 2006.

(51) Int. Cl. A61B 5/00; G01N 33/48

(52) U.S. Cl. 600/300; 702/19

(57) ABSTRACT

A system, method and computer program product for predicting osteoarthritis pain based on changes in barometric pressure and ambient temperature, including inputting average temperature and barometric pressure values; calculating based on the input average temperature and barometric pressure values a pain difference value; interpreting the calculated pain difference value to determine one of a plurality of increasing pain or decreasing pain values; and displaying the determined one of the plurality of the increasing pain or decreasing pain values.

### RELATED APPLICATION DATA

This application claims the benefit of and priority to U.S. Provisional Patent Application Ser. No. 60/820,358 of McAlindon et al., entitled “SYSTEM AND METHOD FOR PREDICTING OSTEOARTHRITIS PAIN BASED ON CHANGE IN BAROMETRIC PRESSURE, AND AMBIENT TEMPERATURE,” filed Jul. 26, 2006, the entire disclosure of which is hereby incorporated by reference herein.

### BACKGROUND OF THE INVENTION

This invention was made with U.S. Government support under NIH/NIAMS Grant (or Contract) No. P60 AR47785, including an initial grant from the Arthritis Foundation and subsequent support from the National Library of Medicine Grant (or Contract) No. RO-1 LM06856-01. The Government has certain rights to this invention.

### FIELD OF THE INVENTION

The present invention generally relates to medical systems and methods, and more particularly to a method and system for predicting osteoarthritis pain based on changes in barometric pressure and ambient temperature.

The present invention includes the use of various technologies described in the references identified in the following LIST OF REFERENCES by the author(s) and year of publication and cross-referenced throughout the specification by reference to the respective number, in brackets, of the reference:

List of References

[1]. Laborde J M, Dando W A, Powers M J. Influence of weather on osteoarthritics. Soc Sci Med 1986;23(6):549-54.

[2]. von Mackensen S, Hoeppe P, Maarouf A, Tourigny P, Nowak D. Prevalence of weather sensitivity in Germany and Canada. Int J Biometeorol 2005;49(3):156-66.

[3]. Quick D C. Joint pain and weather. A critical review of the literature. Minn Med 1997;80(3):25-9.

[4]. Redelmeier D A, Tversky A. On the belief that arthritis pain is related to the weather. Proc Natl Acad Sci U S A 1996;93(7):2895-6.

[5]. McAlindon T, Formica M, LaValley M, Lehmer M, Kabbara K. Effectiveness of glucosamine for symptoms of knee osteoarthritis: results from an internet-based randomized double-blind controlled trial. Am J Med 2004; 117(9):643-9.

[6]. McAlindon T, Formica M, Kabbara K, LaValley M, Lehmer M. Conducting clinical trials over the internet: feasibility study. Bmj 2003;327(7413):484-7.

[7]. Hollander J L. Whether weather affects arthritis. J Rheumatol 1985;12(4):655-6.

[8]. Altman R D. Criteria for the classification of osteoarthritis of the knee and hip. Scand J Rheumatol Suppl 1987;65:31-9.

[9]. Bellamy N, Campbell J, Stevens J, Pilch L, Stewart C, Mahmood Z. Validation study of a computerized version of the Western Ontario and McMaster Universities VA3.0 Osteoarthritis Index. J Rheumatol 1997;24(12):2413-5.

[10]. Tubach F, Ravaud P, Baron G, Falissard B, Logeart I, Bellamy N, et al. Evaluation of clinically relevant changes in patient reported outcomes in knee and hip osteoarthritis: the minimal clinically important improvement. Ann Rheum Dis 2005;64(1):29-33.

[11]. Ehrich E W, Davies G M, Watson D J, Bolognese J A, Seidenberg B C, Bellamy N. Minimal perceptible clinical improvement with the Western Ontario and McMaster Universities osteoarthritis index questionnaire and global assessments in patients with osteoarthritis. J Rheumatol 2000;27(11):2635-41.

[12]. Guedj D, Weinberger A. Effect of weather conditions on rheumatic patients. Ann Rheum Dis 1990;49(3):158-9.

[13]. Sibley J T. Weather and arthritis symptoms. J Rheumatol 1985;12(4):707-10.

[14]. Wilder F V, Hall B J, Barrett J P. Osteoarthritis pain and weather. Rheumatology (Oxford) 2003;42(8):955-8.

[15]. Strusberg I, Mendelberg R C, Serra H A, Strusberg A M. Influence of weather conditions on rheumatic pain. J Rheumatol 2002;29(2):335-8.

[16]. Wingstrand H, Wingstrand A, Krantz P. Intracapsular and atmospheric pressure in the dynamics and stability of the hip. A biomechanical study. Acta Orthop Scand 1990;61(3):231-5.

[17]. Compression Pains. U.S. Navy Diving Manual. Reviison 4 ed: Naval Sea Systems Command, 1999:3-45.

[18]. Golde B. New clues into the etiology of osteoporosis: the effects of prostaglandins (E2 and F2 alpha) on bone. Med Hypotheses 1992;38(2):125-31.

[19]. Osler W. The principles and practice of medicine: designed for the use of practitioners and students of medicine/by William Osler (1892). Special Edition ed. Birmingham: Classics of Medicine Library, 1978.

The entire contents of each reference listed in the LIST OF REFERENCES, are incorporated herein by reference.

### DISCUSSION OF THE BACKGROUND

People with arthritis frequently assert with conviction that weather conditions influence the severity of their pain. ^{[1]} s of individuals with rheumatic disorders show that between one and two-thirds believe that their symptoms are weather-sensitive.^{[2]} However, a consistent relationship between joint pain and weather factors has been strikingly difficult to prove. For example, a recent systematic review of 16 studies of joint pain and weather, found no consensus on the issue.^{[3]} As a result, the medical community has generally viewed the belief as due to psychological misattribution.^{[3, 4]} On the other hand, studies of this question face serious methodological obstacles inherent to evaluating a relationship between a subjective outcome and a universally apparent exposure, especially in the face of firmly-held convictions. As pointed out, these limitations are widely represented among the body of current evidence.^{[3]} Particular problems include disclosure of the study hypothesis to participants, small numbers of participants, and study samples heterogeneous with respect to rheumatic disorders and observation periods.^{[3]} Also, the studies were usually performed at single sites, resulting in limited geographic and meteorologic variability.

### SUMMARY OF THE INVENTION

Therefore, there is a need for a method and system that addresses the above and other problems. The above and other problems are addressed by the exemplary embodiments of the present invention, which provide a method and system for predicting osteoarthritis pain based on changes in barometric pressure and ambient temperature. People with arthritis frequently assert that change in the weather influences their pain, a conviction that physicians generally disbelieve. An online national clinical trial of knee osteoarthritis provided an opportunity to study the influence of short-term change in meteorologic exposures in a fashion that minimizes biases inherent to studies of pain and weather. The exemplary embodiments of the present invention determine that short-term weather parameters, and change in weather parameters, influence the severity of knee pain among people with knee osteoarthritis. A study, accordingly to the exemplary embodiments, evaluates pain in osteoarthritis as the dependent variable in a longitudinal analysis of bi-weekly pain reports collected during a 3-month observation period. Independent variables included daily averages and change in temperature, barometric pressure, dewpoint, precipitation and relative humidity obtained from the weather station closest to each participant. A longitudinal mixed-model random effects analysis with a first-order autoregressive error structure is employed to test for associations while accounting for within-patient correlation. An Internet-based observation study of geographically-dispersed individuals within the U.S. participating in a 3-month clinical trial of glucosamine for knee osteoarthritis is performed. Two hundred participants with confirmed knee osteoarthritis with mean age 60 yrs (standard deviation (s.d.) 9.4), 64% female, mean body mass index 32.5 kg/m² (s.d. 8.4), baseline WOMAC pain score 9.0 (s.d. 3.4), 10.5% African-American or Hispanic are used for the study. The WOMAC pain subscale is administered every two weeks. According to the exemplary embodiments, there are significant and consistent associations of pressure change and ambient temperature with pain severity. With adjustment for age, gender, body mass index, non-steroidal anti-inflammatory drug use, opiate use, and prior pain score, the coefficient for change in barometric pressure was 1.0 (p=0.04), for ambient temperature was −0.01 (p=0.004) and for ambient dewpoint was −0.01 (p=0.02). In mutually adjusted models, there were persistent significant effects from ambient temperature (coefficient=−0.010, 95% confidence limits −0.017−−0.003, p=0.004) and change in barometric pressure (coefficient =1.14, 95% confidence limits 0.15−2.13, p=0.02), but not dewpoint (which was highly correlated with temperature). Both change in barometric pressure and ambient temperature had similar standardized regression coefficients (0.16 and −0.18 respectively). No significant interactions between change in barometric pressure, ambient temperature, or dewpoint using interaction terms entered in the regression models, nor between these variables and age or radiographic severity were found. Use of one-day averages for ambient temperature generated similar mutually adjusted results (coefficient for ambient temperature −0.010, p=0.005; coefficient for change in barometric pressure 1.06, p=0.04). Accordingly, the exemplary embodiments include recognition that rising barometric pressure and colder ambient temperature are independently, associated with greater osteoarthritis knee pain severity and, advantageously, a method and system for predicting osteoarthritis pain based on changes in barometric pressure and ambient temperature are provided.

Accordingly, in exemplary aspects of the present invention there is provided a system, method and computer program product for predicting osteoarthritis pain based on changes in barometric pressure and ambient temperature, including inputting average temperature and barometric pressure values; calculating based on the input average temperature and barometric pressure values a pain difference value; interpreting the calculated pain difference value to determine one of a plurality of increasing pain or decreasing pain values; and displaying the determined one of the plurality of the increasing pain or decreasing pain values

Still other aspects, features, and advantages of the present invention are readily apparent from the following detailed description, by illustrating a number of exemplary embodiments and implementations, including the best mode contemplated for carrying out the present invention. The present invention is also capable of other and different embodiments, and its several details can be modified in various respects, all without departing from the spirit and scope of the present invention. Accordingly, the drawings and descriptions are to be regarded as illustrative in nature, and not as restrictive.

### BRIEF DESCRIPTION OF THE DRAWINGS

The embodiments of the present invention are illustrated by way of example, and not by way of limitation, in the figures of the accompanying drawings and in which like reference numerals refer to similar elements and in which:

FIG. 1 illustrates the location of geographically-dispersed individuals within the U.S. participating in a 3-month clinical trial of glucosamine for knee osteoarthritis, according to the exemplary embodiments;

FIG. 2A illustrates an exemplary coefficient matrix of change in pain scores versus change in pressure and temperature, according to the exemplary embodiments;

FIG. 2B illustrates an exemplary coefficient matrix of change in pain scores versus change in pressure and temperature, according to further exemplary embodiments;

FIG. 3A illustrates an exemplary summary of quantiles across weather data, according to the exemplary embodiments;

FIG. 3B illustrates an exemplary graph of change in pain score versus density, according to the exemplary embodiments; and

FIG. 4 illustrates an exemplary algorithm for predicting osteoarthritis pain based on changes in barometric pressure and ambient temperature, according to the exemplary embodiments.

### DETAILED DESCRIPTION OF THE PREFERRED EMBODIMENTS

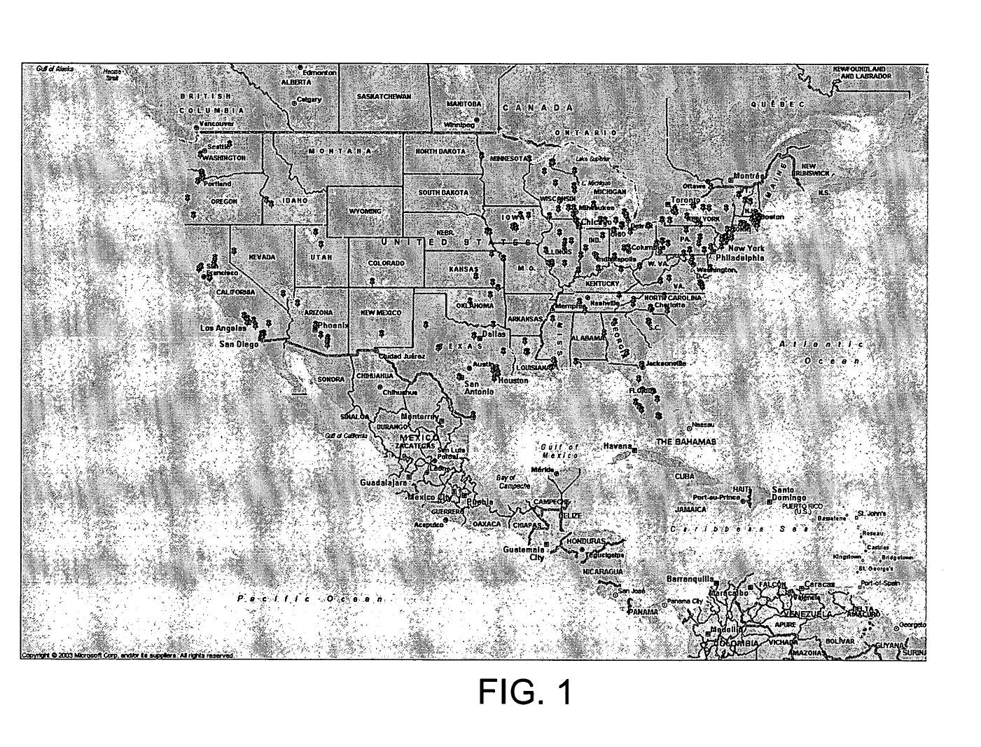

Referring now to the drawings, wherein like reference numerals designate identical or corresponding parts throughout the several views, and more particularly to FIG. 1 thereof, there is illustrated the location of geographically-dispersed individuals within the U.S. participating in a 3-month clinical trial of glucosamine for knee osteoarthritis, according to the exemplary embodiments. Accordingly, the present invention is based on a completed internet-based clinical trial among 205 participants with confirmed knee osteoarthritis and provided a unique opportunity to investigate a relationship between arthritis pain and meteorological conditions in a setting that avoids many of these inherent problems.^{[5],[6]} As shown in FIG. 1, the participants in that trial are geographically-dispersed within the U.S.A. and participated at different times of year. The topic of weather as a research question (for participants or investigators) arose only after completion of the trial, eliminating the opportunity for bias due to disclosure of the study hypothesis. In addition, meteorological data is obtained through an entirely separate prospective collection mechanism. The present invention includes the recognition that most reports of weather influences suggest that these effects operate in the short-term.^{[7]} Therefore, the exemplary embodiments of the present invention test relationships of pain not only with ambient conditions, but also with change in conditions immediately preceding each pain report.

Methods

(i) Sample—The Online Glucosamine Trial

The Online Glucosamine Trial was performed between March 2000 and May 2003, with the methodology and results of the trial reported in detail.^{[5],[6]} Briefly, this is an online deployment of a rigorous randomized placebo-controlled three-month trial of glucosamine sulfate for knee OA symptoms. The participants comprised individuals with knee osteoarthritis classified according to American College of Rheumatology criteria.^{[8]} The study had 205 enrollees with characteristics similar to those of recruits in other, traditional, knee OA trials. ^{[6]} The adherence rate for all scheduled visits is 77%. The outcome of the trial itself is entirely negative, with no difference in pain severity found between the two groups at any time point.^{[6]}

Participants in the Online Glucosamine Trial who are also eligible for the weather analysis are those for whom could be obtained meteorologic data for seven consecutive days prior to at least two pain reports (n=200). The study was approved by the Institutional Review Boards at Boston University School of Medicine.

(ii) Pain Assessments

The primary outcome measure is the pain subscale of the WOMAC questionnaire (Likert version)^{[9]} administered every two weeks over the internet for a total of seven assessments. This 5-item inventory provides a score with range 0-20 reflecting level of pain experienced during different activities of daily life. Since the statistical approach employed a prior pain report in order to adjust for the intra-participant correlation, the associations between pain and climatic parameters are assessed at visits two through seven.

(iii) Acquisition of Meteorologic Data

The most proximate data-collecting weather station to each participant is identified using the search tool provided by the National Oceanographic and Atmospheric Administration website. Next, the local daily average values for temperature, barometric pressure, dewpoint and precipitation for every participant, for each day of their participation in the trial is obtained. The relative humidity from temperature and dewpoint according to the following formula is computed:

Relative Humidity≈100((112−0.1T+Td)/(112+0.9T))^{[8]}

where T=temperature in degrees Celsius and Td=dewpoint temperature in degrees Celsius.

(iv) Exposure Definitions

The exposure parameters are analyzed in two ways (i) as short-term ambient values and (ii) as change values. The short-term ambient values include the means of the daily averages for a) temperature, b) barometric pressure, c) dewpoint, d) precipitation and (e) relative humidity over one- and three-day periods prior to each visit. The change in weather parameters is computed as the difference in each meteorologic parameter between the day prior to each visit and the day of the visit itself.

Non-meteorological exposures in the analyses included age, gender, body mass index, and use at any time in the trial of non-steroidal anti-inflammatory drugs or opiates.

(v) Analytic Approach

A longitudinal mixed-model analysis is employed to test for associations between the meteorologic exposures and knee pain severity, a technique that permits full use of available data while controlling for internal correlations and other covariates. This approach treats each pain report from each participant as a separate observation and adjusts for within-participant correlations and the correlation with the prior pain report. Subjects are treated as random effects so that the analysis can be adjusted to each individual's own pain levels. A first-order autoregressive error structure accounted for within-patient correlation.

While there is no treatment effect evident in the data, a time-trend is observed in pain scores possibly due to regression to the mean. This is adjusted for using a logarithmic function of time of visit as a covariate in the regression models.

In the multivariate models, time from baseline, pain score at the prior visit, and potential confounders (age, gender, body mass index, non-steroidal anti- inflammatory drug use, and opiate-use) are adjusted. Three approaches to deal with missing pain scores are tested—use of baseline pain, a linear interpolation and last pain score carried forwards. Comparison of these approaches using the Bayesian Information Criterion indicated that the linear interpolation approach functioned best. Therefore, that approach is employed in the models. The independent effects of the ambient and change values for the meteorologic parameters in separate and mutually-adjusted models are explored, and the relationships between the meteorological exposures are tested using interaction terms. Finally, stratified analyses is performed to test for non-linearity of temperature effects, and influence of disease severity, including testing for interactions among the meteorological exposures, and between these and other covariates, such as age and osteoarthritis severity, using interaction terms in the multivariate models.

Results

Data from 200 participants are eligible for this analysis. Their mean age is 60 years (s.d. 9.4), 64% are female, they have a mean body mass index of 32.5 kg/m² (s.d. 8.4), mean baseline WOMAC pain score of 9.0 (s.d. 3.4). They provided a total of 935 pain reports, 79% of the total possible. The absent data resulted from missing pain reports (n=265) or missing weather information (n=72).

The geographic dispersion of the sample is illustrated in FIG. 1. The participants are represented by 114 weather stations, 96% of which are within one mile of each participant's residence. The maximum distance is ‘within 50 miles’ and occurs for three participants.

The range and variability of the meteorologic exposures are described in Table 1. Three of these, change in barometric pressure, and ambient temperature and ambient dewpoint, are associated with pain severity in the multivariate models (Table 2). With adjustment for age, gender, body mass index, non-steroidal anti-inflammatory drug use, opiate use, and prior pain score, the coefficient for change in barometric pressure was 1.0 (p=0.04), for ambient temperature was −0.01 (p=0.004) and for ambient dewpoint was −0.01(p=0.02). In mutually adjusted models, there were persistent significant effects from ambient temperature (coefficient=−0.010, 95% confidence limits −0.017−−0.003, p=0.004) and change in barometric pressure (coefficient=1.14, 95% confidence limits 0.15−2.13, p=0.02), but not dewpoint (which was highly correlated with temperature). Both change in barometric pressure and ambient temperature had similar standardized regression coefficients (0.16 and −0.18 respectively). No significant interactions between change in barometric pressure, ambient temperature, or dewpoint using interaction terms entered in the regression models, nor between these variables and age or radiographic severity were found. Use of one-day averages for ambient temperature generated similar mutually adjusted results (coefficient for ambient temperature −0.010, 95% confidence interval=−0.016 to −0.003, p=0.005; coefficient for change in barometric pressure 1.06, 95% confidence interval=0.07 to 2.0, p=0.04).

Discussion

A study of 200 people with knee osteoarthritis participating in a nationwide online clinical trial suggests that both change in barometric pressure and ambient temperature influence severity of knee pain. This study presented a unique opportunity to test the meteorological hypothesis in a way that reduced or eliminated many of the biases present in previous studies of this question. In the first place, the participants are geographically-dispersed and participated at different times of year, generating greater opportunity for weather exposure variability. Further, the topic of weather as a research question arose only after completion of the trial, minimizing the opportunity for bias due to disclosure of the study hypothesis. The WOMAC pain subscale, a well-validated instrument,^{[9]} to prospectively collect knee pain data is employed. Prospectively-collected meteorological data is obtained through an entirely separate mechanism and analyzed with modem statistical mixed-model methods.

The above factors are salient because interpretation of prior studies in this field is obfuscated by their methodological limitations and problems of inference inherent to testing the relationship of pain to observable meteorological conditions, especially in the face of strongly held convictions. Quick identified a large number of such problems among prior studies of joint pain and weather scrutinized in his critical review.^{[3]} These flaws included failures to (i) keep participants uninformed about the topic of the study and weather reports, (ii) test effects of changes in the weather (iii) observe participants for periods sufficient to include seasonal variations. In addition, the previous studies focused on varying patient populations (among whom weather effects might operate differently), had limited geographic dispersion of participants (limiting weather variability), and used heterogeneous outcome measures. These issues could account for inconclusive and apparently contradictory results among the studies scrutinized by Quick.^{[3]}

The two significant exposure variables had similar effects in the models based on their standardized coefficients (0.16 for change in barometric pressure and −0.18 for ambient temperature), but the magnitude of their effects was small in relation to changes in pain that are considered to be clinically significant. For example, change in barometric pressure had an effect on knee pain, commensurate with an increase in knee pain score of 1.0 for a rise of 1 inch of mercury in barometric pressure. The coefficient for ambient temperature was equivalent to an increase in knee pain score of 0.1 for each 10 degree Fahrenheit fall in temperature. It is discovered odd that effects below a magnitude considered to be clinically significant^{[10],[11]} should be so consistent in the results. This led to exploration of whether the importance of this exposure might reside in an indirect effect or interaction, but interaction terms in the multivariate models revealed no evidence of an interaction of ambient temperature with the other meteorologic parameters or with age or disease severity. Similarly, no evidence is found that the effect operated differently within rage strata. An alternative explanation may relate to the temperature differences between the outdoor and indoor climate. A tendency to remain indoors, especially during extremes of outdoor temperatures, could limit the opportunity of this exposure to exert an effect. Thus, during cold weather, the effect might only manifest among individuals who venture outdoors. Even if the effect among the individuals who venture outdoors was large, the inherent misclassification consequent on combining their data with those who remained indoors would be to attenuate the aggregated effect computed in the statistical models. Because information on participants' activities was not available, such possibility could not be examined directly.

The results also suggest that rising barometric pressure is associated with increased pain. The interpretation of this association in terms of observable weather is not straightforward, though, because the relationship between pressure change and weather events is variable. In general, severe deterioration in weather is often accompanied by marked fluctuations in pressure. A sequence of events in which a rise in pressure precedes or accompanies the development of precipitation could explain the phenomenon in which some people with arthritis believe they can predict adverse developments in the weather.

A possible weakness of the study is that a measurement of affect was not available, so it was not possible to directly explore psychological factors (such as mood) as confounders or mediators of weather effects. However, there are several reasons to doubt that psychological mechanisms could mediate the association of rising barometric pressure with increased pain. Firstly, changes in pressure are not directly perceptible. Secondly, the data was collected prospectively eliminating the opportunity for participants to make retrospective pain assignments that could be biased by the subsequent appearance of bad weather. While participants could theoretically have been noting weather predictions, it is more likely that they would have ascribed increasing pain to falling pressure, in line with the prevalent folklore. Also, variables more likely to have direct psychological effects, such as precipitation or humidity, had no significant influences in the data.

Another limitation in the study is that it was not possible to adjust for analgesics taken on the day of each pain report. Such analgesics might be expected to attenuate any association with pain severity. As such, this could bias the findings towards the null rather than generate spurious associations.

Methodological issues and differences in exposure and outcome definitions complicate direct comparison of the results of previous studies in this field with the present studies. For example, a recent systematic review that used a weighted consensus voting approach favored an association of osteoarihritis pain with high barometric pressure, but none of the contributory studies tested short-term fluctuation in pressure.^{[1],[3],[12],[13]} However, Wilder et al compared pain scores and weather conditions among 154 individuals with osteoarthritis participating in a 2-year exercise intervention study in Florida and noted positive associations with days of ‘rising barometric pressure’ in a subset.^{[14]} Strusberg et al found a correlation of pain with low ambient temperature among people with osteoarthritis but did not examine pressure change.^{[15]}

The results are also apparently at odds with Hollander's early experiments using a controlled climate chamber.^{[7]} These studies suggested that a simultaneous increase in humidity accompanied by a fall in barometric pressure increased pain, swelling and stiffness due to arthritis. However, these experiments included only four individuals with OA, one of whom was not ‘weather sensitive’. The timing of the atmospheric changes, and the short observation periods, may also have limited the external validity of their findings.

The plausibility of the findings is predicated on a biological explanation of how change in barometric pressure, and temperature, might influence osteoarthritis pain. Notably, there is evidence that barometric pressure contributes to joint integrity. Wingstrand et al, in a study of in cadaveric hips, found the intra- articular pressure to be sub-atmospheric in normal situations.^{[16]} When the intra-articular pressure is equilibrated with the atmosphere, the hip joints exhibited 8 mms of subluxation without significant traction. This shows that atmospheric pressure has a physical role in stabilizing the hip joint.^{[16]} Furthermore, they found intra-articular pressures to be elevated in the presence of joint effusion. The direct effect of atmospheric pressure on joint biomechanics could have additional consequences in certain situations, such as joints with effusions or those in which a defect of articular cartilage integrity allows communication between the intracapsular space and the richly innervated subchondral bone and marrow. Such pathology-specific pressure effects might explain the inconsistent results of studies that investigated the influence of weather using heterogeneous samples.

Interestingly, joint pains during compression are well recognized among divers, especially saturation divers.^{[7]} The mechanism is unknown, but is conjectured to result from the sudden increase in tissue gas tension surrounding the joints causing fluid shifts and interfering with joint lubrication.

Cold temperatures might also affect joint pain through a number of mechanisms.^{[3]} Temperature could have direct effects on the compliance of peri-articular structures and viscosity of synovial fluid, and indirect effects on inflammatory mediators through influences on capillary permeability.^{[18]}

The data corroborate the general assertions by people with osteoarthritis that weather conditions influence their pain. Agreement on this issue may enhance physician-patient interactions and help them better understand and manage fluctuations in arthritis pain. While the therapeutic implications of the findings can be further developed, they may already help people with osteoarthritis plan their lives. Indeed, Osler's advice for arthritis sufferers in 1892 may have been partly correct when he asserted that “Many cases are greatly helped by prolonged residence in southern Europe or southern California. Rich patients should always be encouraged to winter in the south and in this way avoid cold, damp weather.”^{[19]}

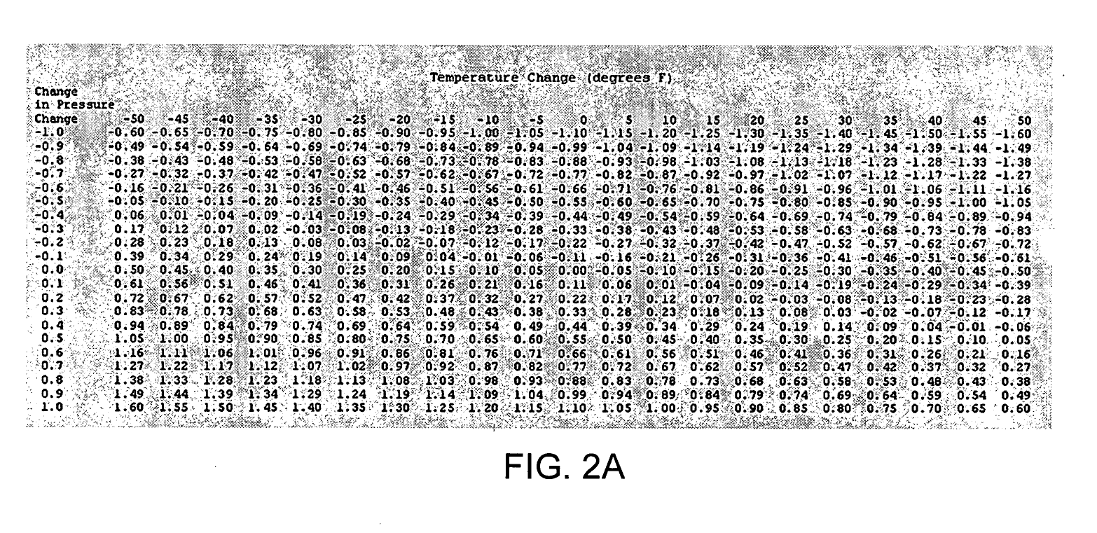

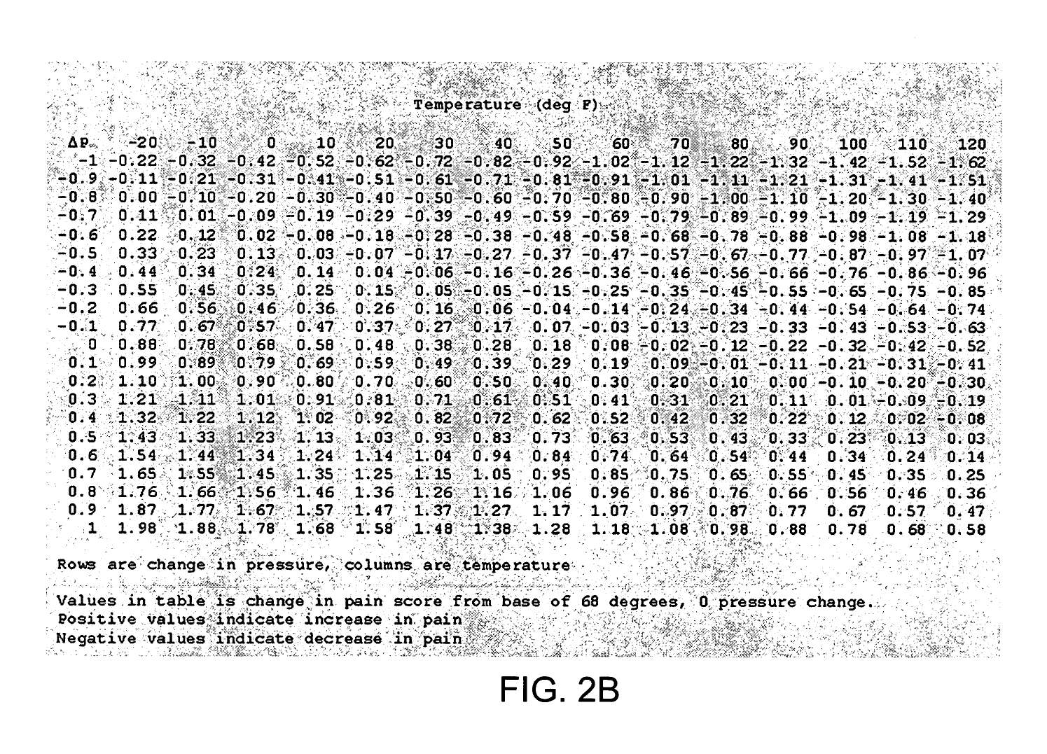

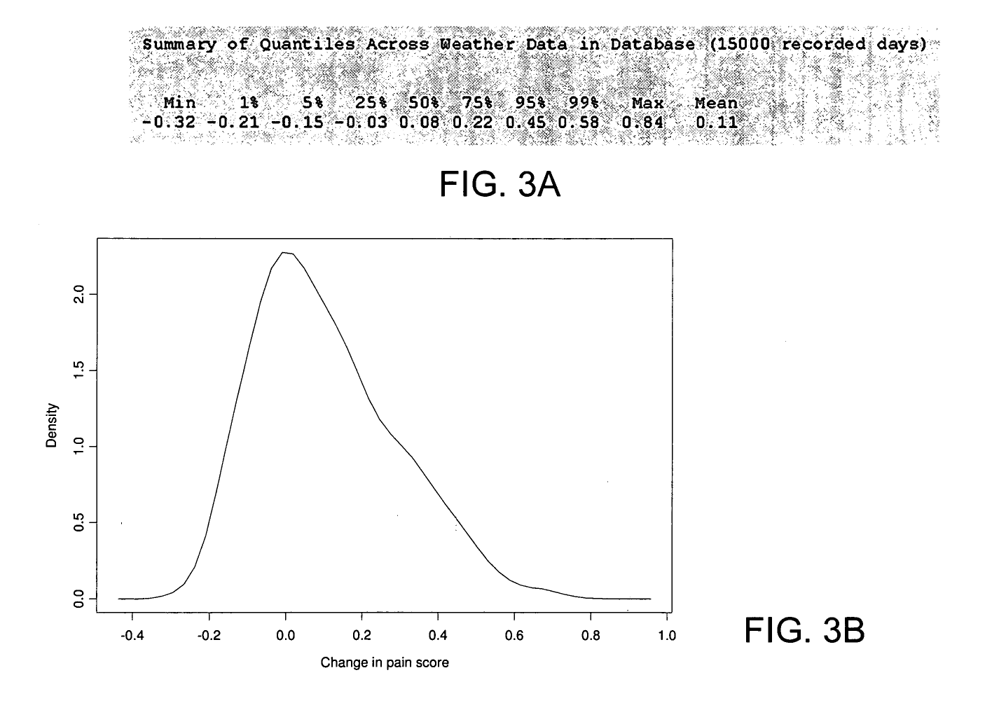

Advantageously, the clinical significance of the invention according to the exemplary embodiments is that people with arthritis frequently assert that change in the weather influences their pain, a conviction that physicians generally disbelieve. The analysis of the exemplary embodiments of individuals with knee osteoarthritis confirms effects of weather on knee pain severity and suggests that this is mediated by short-term rise in barometric pressure and colder ambient temperature. Accordingly, FIG. 2B illustrates an exemplary coefficient matrix of change in pain scores versus change in pressure and temperature, according to the exemplary embodiments. FIG. 3A illustrates an exemplary summary of quantiles across weather data, according to the exemplary embodiments. FIG. 3B illustrates an exemplary graph of change in pain score versus density, according to the exemplary embodiments.

Table 1 below illustrates the distribution of meteorological exposures in the study sample.

TABLE 1
Distribution of meteorological exposures in the study sample

| | | |
|---|---|---|
| mean | s.d. | range |

Ambient weather *

| | | | |
|---|---|---|---|
| Temperature (degrees Fahrenheit) | 56.9 | 15.4 | −7.3-95.7 |
| Barometric Pressure (inches mercury) | 29.1 | 1.0 | 24.6-30.4 |
| Dewpoint (degrees Fahrenheit) | 46.2 | 14.3 | −6.6-76.4 |
| Precipitation (inches) | 0.09 | 0.12 | 0-1.92 |
| Relative Humidity | 68.2 | 14.3 | 18.2-98.6 |

Weather change **

| | | | |
|---|---|---|---|
| Temperature (degrees Fahrenheit) | −0.13 | 3.0 | −22-28 |
| Barometric Pressure (inches mercury) | −0.008 | 0.06 | −0.58-0.51 |
| Dewpoint (degrees Fahrenheit) | 0.37 | 3.4 | −29.0-24.0 |
| Precipitation (inches) | 0.004 | 0.15 | −3.1-2.4 |
| Relative Humidity | 0.7 | 12.3 | −48.1-47.0 |

* Average of within-participant means over the 3-day period prior to each pain report
** Change between the day prior to each pain report and the day of the pain report

Table 2 below shows the meteorologic exposures and knee pain in a multivariable analyses.

TABLE 2
Meteorologic exposures & knee pain: multivariable analyses

| | Ambient weather* | | Weather change** | |
|---|---|---|---|---|
| | coefficient† | p-value | coefficient† | p-value |
| Temperature (degrees | −0.01 | 0.004 | 0.01 | 0.3 |
| Fahrenheit) | | | | |
| Barometric Pressure | 0.03 | 0.7 | 1.0 | 0.04 |
| (inches mercury) | | | | |
| Dewpoint (degrees | −0.01 | 0.02 | 0.004 | 0.7 |
| Fahrenheit) | | | | |
| Precipitation (inches) | −0.5 | 0.2 | −0.1 | 0.7 |
| Relative Humidity | 0.001 | 0.8 | −0.003 | 0.6 |

*Average of within-participant means over the 3-day period prior to each pain report
**Change between the day prior to each pain report and the day of the pain report
†Adjusted for regression to the mean, prior pain score, age, sex, body mass index, NSAID use, and opiate-use

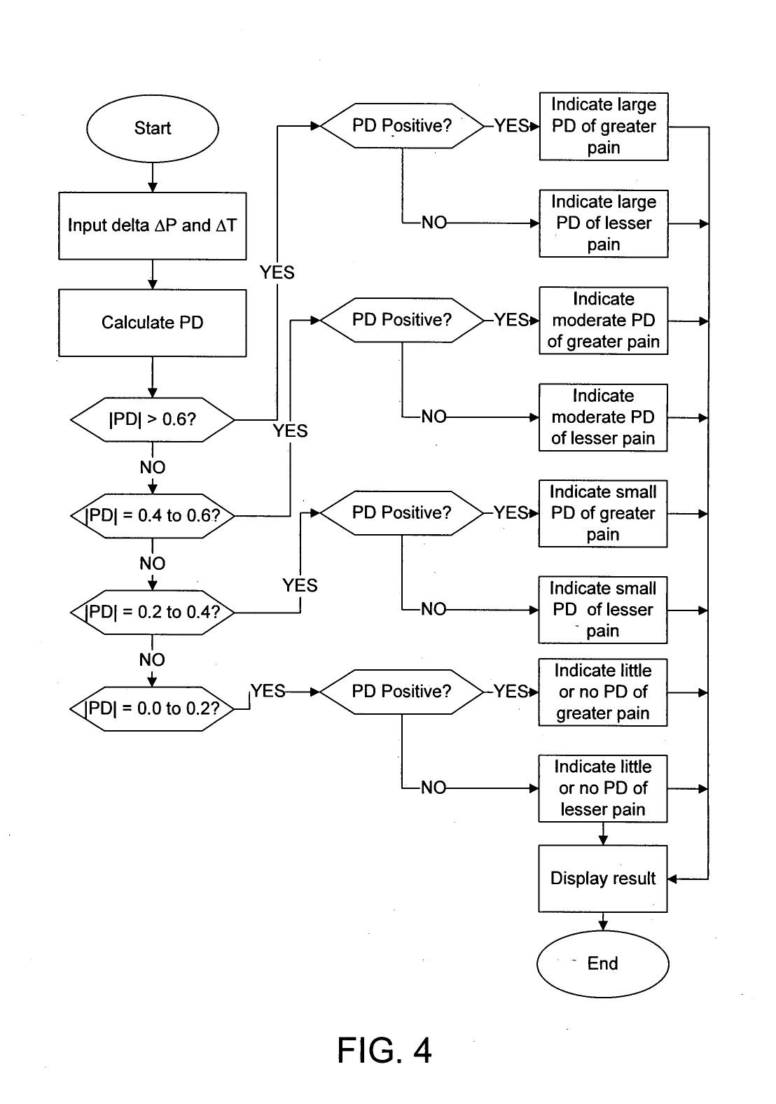

Accordingly, the following exemplary algorithms, as illustrated in FIG. 4, can be employed for predicting osteoarthritis pain based on changes in barometric pressure and ambient temperature. For example, an exemplary algorithm (e.g., based on FIG. 2A) for determining arthritis pain level today relative to yesterday can be given by:

Pain difference=([avg. temp* today−avg. temp yesterday]×−0.01)+([pressure change** today−pressure change yesterday]×1.06)

*Avg. temp refers to the 24-hour daily average temperature in degrees F. ** pressure change is defined as the difference in 24-hour average ambient pressure (inches Hg) between the index day and the day preceding it.

An exemplary algorithm (e.g., based on FIG. 2A) for a forecast of arthritis pain level tomorrow relative to today can be given by:

Pain difference=([avg. temp tomorrow−avg. temp today]×−0.01) +([pressure change tomorrow−pressure change today]×1.06)

For example, determining an arthritis pain level today relative to a balmy 68 degree F day with no pressure change (e.g., based on FIG. 2B) can be given by:

Pain difference=([avg. temp today−68]×−0.01)+([pressure change today]×1.06)

An exemplary interpretation of pain difference values based on the frequency distribution of pain differences is shown in Table 3, as follows:

TABLE 3
Meteorologic exposures & knee pain: multivariable analyses

| Absolute Value | Interpretation |
|---|---|
| >0.6 | large pain difference |
| 0.4-0.6 | moderate pain difference |
| 0.2-0.4 | small pain difference |
| 0-0.2 | little or no pain difference |

* Positive values indicate relatively greater pain
** Negative values indicate relatively less pain

Advantageously, the exemplary embodiments can employ the above algorithms to provide a “pain index” score to arthritis sufferers. The pain index can be either an absolute scale, or can report pain change relative to a previous day. The index can be based on a whole number system, a fractional number system, a graphical indication of rising or falling pain levels (such as arrows or plus signs), a graphical indication of absolute pain (such as smiling or frowning faces), a color index in which one end of the color spectrum is indicative of high pain levels and the other end of the spectrum is indicative of low pain levels, and the like.

Advantageously, the pain index can allow arthritis sufferers to plan activities according to their projected pain levels, and to plan to take medication to prevent or reduce pain when levels are expected to be high. In addition, the pain index can be used or reported by any suitable publication, online or print, that reports weather, such as golf, ski, tennis, and other recreation-related websites, newspapers, airline websites, weather websites, arthritis-related journals and websites, and the like.

Advantageously, the pain index can be used as an indication of pain caused by arthritis, and more preferably as an indication of pain caused by osteoarthritis, and most preferably as an indication of pain caused by osteoarthritis of the knee.

Advantageously, the exemplary algorithms can be differentiated from other arthritis/weather indices in being based on more reliable data as well as in the quantitative components of the computations thereof.

The above-described devices and subsystems of the exemplary embodiments can include, for example, any suitable servers, workstations, PCs, laptop computers, PDAs, Internet appliances, handheld devices, cellular telephones, wireless devices, other devices, and the like, capable of performing the processes of the exemplary embodiments. The devices and subsystems of the exemplary embodiments can communicate with each other using any suitable protocol and can be implemented using one or more programmed computer systems or devices.

One or more interface mechanisms can be used with the exemplary embodiments, including, for example, Internet access, telecommunications in any suitable form (e.g., voice, modem, and the like), wireless communications media, and the like. For example, employed communications networks or links can include one or more wireless communications networks, cellular communications networks, G3 communications networks, Public Switched Telephone Network (PSTNs), Packet Data Networks (PDNs), the Internet, intranets, a combination thereof, and the like.

It is to be understood that the devices and subsystems of the exemplary embodiments are for exemplary purposes, as many variations of the specific hardware used to implement the exemplary embodiments are possible, as will be appreciated by those skilled in the relevant art(s). For example, the functionality of one or more of the devices and subsystems of the exemplary embodiments can be implemented via one or more programmed computer systems or devices.

To implement such variations as well as other variations, a single computer system can be programmed to perform the special purpose functions of one or more of the devices and subsystems of the exemplary embodiments. On the other hand, two or more programmed computer systems or devices can be substituted for any one of the devices and subsystems of the exemplary embodiments. Accordingly, principles and advantages of distributed processing, such as redundancy, replication, and the like, also can be implemented, as desired, to increase the robustness and performance of the devices and subsystems of the exemplary embodiments.

The devices and subsystems of the exemplary embodiments can store information relating to various processes described herein. This information can be stored in one or more memories, such as a hard disk, optical disk, magneto-optical disk, RAM, and the like, of the devices and subsystems of the exemplary embodiments. One or more databases of the devices and subsystems of the exemplary embodiments can store the information used to implement the exemplary embodiments of the present inventions. The databases can be organized using data structures (e.g., records, tables, arrays, fields, graphs, trees, lists, and the like) included in one or more memories or storage devices listed herein. The processes described with respect to the exemplary embodiments can include appropriate data structures for storing data collected and/or generated by the processes of the devices and subsystems of the exemplary embodiments in one or more databases thereof.

All or a portion of the devices and subsystems of the exemplary embodiments can be conveniently implemented using one or more general purpose computer systems, microprocessors, digital signal processors, micro-controllers, and the like, programmed according to the teachings of the exemplary embodiments of the present inventions, as will be appreciated by those skilled in the computer and software arts. Appropriate software can be readily prepared by programmers of ordinary skill based on the teachings of the exemplary embodiments, as will be appreciated by those skilled in the software art. Further, the devices and subsystems of the exemplary embodiments can be implemented on the World Wide Web. In addition, the devices and subsystems of the exemplary embodiments can be implemented by the preparation of application-specific integrated circuits or by interconnecting an appropriate network of conventional component circuits, as will be appreciated by those skilled in the electrical art(s). Thus, the exemplary embodiments are not limited to any specific combination of hardware circuitry and/or software.

Stored on any one or on a combination of computer readable media, the exemplary embodiments of the present inventions can include software for controlling the devices and subsystems of the exemplary embodiments, for driving the devices and subsystems of the exemplary embodiments, for enabling the devices and subsystems of the exemplary embodiments to interact with a human user, and the like. Such software can include, but is not limited to, device drivers, firmware, operating systems, development tools, applications software, and the like. Such computer readable media further can include the computer program product of an embodiment of the present inventions for performing all or a portion (if processing is distributed) of the processing performed in implementing the inventions. Computer code devices of the exemplary embodiments of the present inventions can include any suitable interpretable or executable code mechanism, including but not limited to scripts, interpretable programs, dynamic link libraries (DLLs), Java classes and applets, complete executable programs, Common Object Request Broker Architecture (CORBA) objects, and the like. Moreover, parts of the processing of the exemplary embodiments of the present inventions can be distributed for better performance, reliability, cost, and the like.

As stated above, the devices and subsystems of the exemplary embodiments can include computer readable medium or memories for holding instructions programmed according to the teachings of the present inventions and for holding data structures, tables, records, and/or other data described herein. Computer readable medium can include any suitable medium that participates in providing instructions to a processor for execution. Such a medium can take many forms, including but not limited to, non-volatile media, volatile media, transmission media, and the like. Non-volatile media can include, for example, optical or magnetic disks, magneto-optical disks, and the like. Volatile media can include dynamic memories, and the like. Transmission media can include coaxial cables, copper wire, fiber optics, and the like. Transmission media also can take the form of acoustic, optical, electromagnetic waves, and the like, such as those generated during radio frequency (RF) communications, infrared (IR) data communications, and the like. Common forms of computer-readable media can include, for example, a floppy disk, a flexible disk, hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read.

While the present inventions have been described in connection with a number of exemplary embodiments, and implementations, the present inventions are not so limited, but rather cover various modifications, and equivalent arrangements, which fall within the purview of prospective claims.

What is claimed is:

1. A method for predicting osteoarthritis pain based on changes in barometric pressure and ambient temperature, comprising:
inputting average temperature and barometric pressure values;
calculating based on the input average temperature and barometric pressure values a pain difference value;
interpreting the calculated pain difference value to determine one of a plurality of increasing pain or decreasing pain values; and
displaying the determined one of the plurality of the increasing pain or decreasing pain values.

2. The method of claim 1, further comprising implementing the method with one or more hardware and software devices.

3. The method of claim 1, further comprising implementing the method with one or more computer readable instructions embedded on a computer readable medium and configured to cause one or more computer processors to perform the steps of the method.

4. A system for predicting osteoarthritis pain based on changes in barometric pressure and ambient temperature, comprising:
means for inputting average temperature and barometric pressure values;
means for calculating based on the input average temperature and barometric pressure values a pain difference value;
means for interpreting the calculated pain difference value to determine one of a plurality of increasing pain or decreasing pain values; and
means for displaying the determined one of the plurality of the increasing pain or decreasing pain values.

5. The system of claim 4, wherein the system is implemented with one or more hardware and software devices.

6. A computer program product for predicting osteoarthritis pain based on changes in barometric pressure and ambient temperature and including one or more computer readable instructions embedded on a computer readable medium and configured to cause one or more computer processors to perform the steps of:
inputting average temperature and barometric pressure values;
calculating based on the input average temperature and barometric pressure values a pain difference value;
interpreting the calculated pain difference value to determine one of a plurality of increasing pain or decreasing pain values; and
displaying the determined one of the plurality of the increasing pain or decreasing pain values.
